(19) United States
(12) Patent Application Publication
(10) Pub. No.: US 2021/0212921 A1
(43) Pub. Date: Jul. 15, 2021

(54) Compositions for Topical Treatment

(71) Applicant: RR HEALTH, LLC, Farmington Hills, MI (US)

(72) Inventors: Don Decker, Candler, NC (US); Amanda Vickers, Candler, NC (US)

(21) Appl. No.: 17/058,516

(22) PCT Filed: May 29, 2019
(86) PCT No.: PCT/US2019/034462

§ 371 Date: Nov. 24, 2020

Related U.S. Application Data
(60) Provisional application No. 62/677,625, filed on May 29, 2018.

(51) Int. Cl. A61K 8/92; A61K 8/37; A61K 8/34; A61K 8/49; A61K 8/55; A61Q 19/00

(52) U.S. Cl. CPC A61K 8/922; A61K 8/375; A61K 8/342; A61K 8/347; A61K 2800/24; A61K 8/553; A61Q 19/007; A61K 2800/5922; A61K 8/4946

(57) ABSTRACT

A composition for forming a solid material at a temperature of about 60° F. to about 77° F. comprising an oil phase including one or more oils in an amount of from 30% to 70% by weight of the composition, and one or more alcohols; and an aqueous phase including one or more polyglycerides; wherein the ratio of the aqueous phase to the one or more polyglycerides does not exceed 2.5:1.

### FIELD OF INVENTION

The present teachings relate generally to treatments for skin conditions. More particularly, the present teachings relate to wax-free lip treatments.

### BACKGROUND

Most lip treatment compositions include a wax substance that effectively prevents the lips from absorbing moisture. This makes the treatment of dry, cracked lips challenging in that moisture is needed to relieve these ailments. As a further issue, most lip treatments are limited in their timeline for efficacy and provide relief only upon application while failing to provide continuous healing to damaged lips.

U.S. Pat. Nos. 7,073,965; 7,695,727; and 8,563,048 teach natural lip balm treatments. However, each rely on wax to develop a desirable homogenous and rigid stick-like format.

Based upon these shortcomings, there remains a need for wax-free topical treatments that facilitate moisture to damaged skin while providing healing over an extended period of time.

### SUMMARY OF INVENTION

The present teachings meet the above-mentioned needs by providing a natural (e.g., naturally derived) wax-free, solid, water in oil emulsion formula, where the internal aqueous phase is a microdispersion. The formula may be utilized for the relief of symptoms and/or discomfort related, but not limited to, dry lips, cracked lips or lip fatigue. The composition described herein may be utilized after a performance involving any brass or woodwind musical instrument. The composition described herein may be utilized in a cosmetic formulation, including but limited to a lip stick or lip gloss formulation. As a solid form, water in oil emulsion, this formula can be tailored to deliver a range of ingredients, both water or oil soluble, to the lips. It delivers moisture to (hydrates) the lips, while simultaneously, thru its lipophilic character, creates a water barrier, thereby reducing trans epidermal water loss (TEWL) and increasing slip between the upper and lower lip. Active water and oil soluble ingredients are made available and released to the lip epidermis and dermis on an extended time basis after application thru the physical contact with and rub in by the upper and lower lip and/or the tongue.

The teachings herein are directed to a composition for forming a solid material at a temperature of about 60 to about 77° F. comprising an oil phase including one or more oils in an amount of from 30% to 70% by weight of the composition, and one or more alcohols and an aqueous phase including one or more polyglycerides. The ratio of the aqueous phase to the one or more polyglycerides may not exceed 2.5:1.

The composition may be substantially free of any wax material. The composition may be shaped for application to a user's lips. The oil phase may comprise castor oil. The oil phase may comprise castor oil in an amount of from about 30% to about 65% by weight of the composition. The oil phase may comprise castor oil in an amount of from about 45% to about 60% by weight of the composition. The oil phase may comprise castor oil in an amount of at least 30% by weight of the composition. The oil phase may comprise castor oil in an amount of at least 40% by weight of the composition. The oil phase may comprise an alcohol which may be behenyl alcohol. The oil phase may comprise an alcohol which may be behenyl alcohol in an amount of from about 5% to about 30% by weight of the composition. The oil phase may comprise an alcohol which may be behenyl alcohol in an amount of from about 13% to about 25% by weight of the composition. The oil phase may comprise an alcohol which may be behenyl alcohol in an amount of at least 10% by weight of the composition. The oil phase may comprise an alcohol which may be behenyl alcohol in an amount of at least 15% by weight of the composition.

The oil phase may comprise hempseed butter in an amount of from about 0.05% to about 20% by weight of the composition. The oil phase may comprise hempseed butter in an amount of from about 0.5% to about 15% by weight of the composition. The oil phase may comprise hempseed butter in an amount of from about 0.05% to about 20% by weight of the composition. The oil phase may comprise an antioxidant. The oil phase may comprise butylated hydroxytoluene (BHT). The oil phase may comprise natural plant oil, essential oil, oil soluble vitamins, antioxidants, natural color, natural preservatives, or any combination thereof.

The aqueous phase may comprise from about 0.01% to about 30% by weight of the composition. The aqueous phase may comprise from about 0.05% to about 20% by weight of the composition. The aqueous phase may comprise less than 30% by weight of the composition. The aqueous phase may comprise less than 20% by weight of the composition.

The composition may be substantially homogeneous and substantially rigid at temperatures below 50° C. The composition may have a melt point of at least about 52° C. The composition may have a proximal drop point of at least 55° C.

The one or more alcohols may be selected from fatty or long-chain alcohols, or combinations thereof. The one or more alcohols may be selected from C22 fatty alcohol or behenyl alcohol, or combinations thereof. The one or more alcohols may be mixed with the one or more oils at a temperature of at least about 71° C. The aqueous phase may include one or more oleic, linoleic, or linolenic polyglycerides. The aqueous phase may include one or more oleic, linoleic, or linolenic polyglycerides in an amount of from about 0.5% to about 10% by weight of the composition. The aqueous phase may include one or more oleic, linoleic, or linolenic polyglycerides in an amount of from about 0.8% to about 8% by weight of the composition. The aqueous phase may include a non-ionic surfactant/emulsifier selected from sorbitan sesquioleate, Isolan 17 (polyglyceryl-4-diisostearate/polyhydroxystearate/sebacate, caprylic/capric triglyceride, polyglycerides-3-oleate, diisostearyl polyglyceryl-3 dimer dilinoleate, lecithin, or combinations thereof. The aqueous phase may include sorbitan sesquioleate. The aqueous phase may include a non-ionic surfactant in an amount of about 0.001% to about 8% by weight of the composition. The aqueous phase may include a non-ionic surfactant in an amount of about 0.01% to about 3% by weight of the composition. The non-ionic surfactant may be sorbitan sesquioleate. The ratio of a non-ionic surfactant to the one or more polyglycerides may be from about 1:4 to about 1:10.

The composition may include hempseed butter. The composition may include an antioxidant. The composition may include an antioxidant selected from butylated hydroxytoluene, acai oil, alpha lipoic acid, retinol, vitamin C, coenzyme Q10, isoflavones, polyphenols, or combinations thereof.

The composition may include a combination of castor oil, behenyl alcohol, hempseed butter and butylated hydroxytoluene. The aqueous phase may include one or more of water, glycerin, fruit extracts, Lactobacillus and combinations thereof. The aqueous phase may include one or more of Lactobacillus and Cocos nucifera fruit extract.

The teachings herein are further directed to a method for formulating the composition as described herein, including melting the one or more alcohols in the one or more oils at a temperature of at least about 71° C. The teachings herein include a method for forming a topical treatment comprising preparing an oil phase including mixing one or more oils in an amount of from 30% to 70% by weight of the composition, and one or more alcohols at a temperature of at least 70° C., preparing an aqueous phase including one or more antioxidants and one or more polyglycerides, and combining the oil phase and aqueous phase at a temperature of least 55° C., at least 65° C. or even at least 70° C. The method may include, shaping the final product into a stick-like format. The method may include locating the final product into a tube-shaped dispensing device for use.

The teachings herein are further directed to use of the composition as described herein for forming a lip protectant.

### DETAILED DESCRIPTION OF THE INVENTION

The explanations and illustrations presented herein are intended to acquaint others skilled in the art with the invention, its principles, and its practical application. Accordingly, the specific embodiments of the present disclosure as set forth are not intended as being exhaustive or limiting of the invention. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

The composition described herein is preferably formed as a smooth, homogenous, hard stick (at temperatures of less than about 50° C.), with a high melt point of at least 35° C., at least 45° C. or even at least 55° C.). The composition may have a proximal drop point of at least 40° C., at least 45° C. or even at least 55° C.). The composition may further have a narrow solidification range (e.g., about 1-10° C., about 2-8° C. or even about 4° C.).

This application claims the benefit of the filing date of U.S. Provisional Application Ser. No. 62/677,625, filed May 29, 2018, the contents of that application being incorporated by reference for all purposes.

The composition may include one or more oils, one or more alcohols (e.g., fatty or long-chain alcohols such as C22 fatty alcohol or behenyl alcohol), one or more polyglycerides, one or more hydrocarbons or plant-derived hydrocarbons, one or more antioxidants, and one or more surfactants/emulsifiers. The one or more surfactants/emulsifiers are selected from sorbitan sesquioleate, Isolan 17 (polyglyceryl-4-diisostearate/polyhydroxystearate/sebacate, caprylic/capric triglyceride, polyglycerides-3-oleate, diisostearyl polyglyceryl-3 dimer dilinoleate, lecithin, or combinations thereof. The composition may include plant-derived materials which may be selected from hemp seed oil, squalene, squalane, or combinations thereof.

The composition preferably includes one or any combination of castor oil, behenyl alcohol, oleic/linoleic/linolenic polyglycerides, sorbitan sesquioleate, hempseed oil (e.g., hemp seed butter), butylhydroxytoluene (BHT) and an aqueous phase.

During processing, it is possible that the one or more alcohols are melted in the castor oil (e.g., the oil phase components), with mixing at a temperature of at least about 55° C., at least about 65° C., or even at least about 70° C. The mixture is then cooled with mixing, to a temperature of less than 80° C. or even less than 70° C. After mixing of the oil phase components, the remaining oil soluble ingredients can be added. This constitutes the external oil phase of the finished formula.

During processing, it is also possible that any water soluble ingredients be pre-dissolved in an aqueous solution prior to dispersing into the oleic/linoleic/linolenic polyglycerides and non-ionic surfactants (e.g., the dispersant phase). This mixture constitutes the internal aqueous phase. The aqueous solution may include water, glycerin, fruit extracts, Lactobacillus and combinations thereof. For example, the aqueous solution may include Lactobacillus and Cocos nucifera fruit extract. An example of which is sold under the tradename AMTicide® Coconut, available from Active Micro Technologies, Lincolnton, N.C.

The aqueous dispersion may then be mixed into the oil phase at a temperature of at least about 55° C., at least about 65° C. or even at least about 70° C. The resulting aqueous/oil mixture is cooled with moderate/vigorous mixing, to approximately 60° C. The completed emulsion can then be filled into a desired container, which may be but is not limited to a lip balm stick container.

The oil phase may comprise from about 70% to about 95% by weight, or even from about 80% to about 90%, by weight, of the total formula. The aqueous phase may comprise from about 5% to about 30%, or even about 10% to about 20% by weight of the total formula.

The one or more oils may be present in an amount of from about 30% to about 70%, or even about 47% to 57% by weight of the total formula. The one or more oils may be present in an amount of about 52% by weight of the total formula.

The one or more alcohols may be present in an amount of from about 10% to about 35%, or even about 17% to 23% by weight of the total formula. The one or more alcohols may be present in an amount of about 20% by weight of the total formula.

The one or more hydrocarbons and/or plant-derived materials may be present in an amount of from about 0.05% to about 30%, or even about 2% to 10% by weight of the total formula. The one or more hydrocarbons may be present in an amount of about 6% by weight of the total formula.

The one or more antioxidants may be present in an amount of from about 0.001% to about 5%, or even about 0.1% to 1% by weight of the total formula. The one or more antioxidants may be present in an amount of about 0.5% by weight of the total formula.

The aqueous phase may be present in an amount of from about 0.01% to about 30%, or even about 1% to 15% by weight of the total formula.

The one or more polyglycerides may be present in an amount of from about 0.05% to about 30%, or even about 1.6% to 6% by weight of the total formula. The ratio of aqueous to the one or more polyglycerides preferably does not exceed 2.5:1. However, lower ratios are acceptable, down to 0.5:1.0.

The one or more surfactants/emulsifiers may be present in an amount of from about 0.001% to about 5%, or even about 0.3% to 1.2% by weight of the total formula. The ratio of the one or more surfactants/emulsifiers to the polyglycerides preferably does not exceed 1:4. However, higher ratios are acceptable, up to 1:10.

The formula may include one or more additional lipophilic (oil soluble) components. One or more lipophilic components can be added in an amount of 0.0% to 6.0% by weight of the total formula. These lipophilic components may include, but are not limited to natural plant oils, essential oils, oil soluble vitamins, antioxidants, natural colors and/or natural preservatives.

The formula may include one or more additional hydrophilic (water soluble) components. These water soluble components may be added to the aqueous phase, at a ratio not to exceed 1:5, with the total weight of the water and water soluble ingredients, not to exceed 15% by weight of the total formula.

The present teachings provide for inclusion of a substantially evenly dispersed internal water phase into a solid stick formulation (e.g., a stick that is solid at typical room temperature and use conditions). This format allows for the delivery of water soluble active ingredients (humectants) to the specific target site (lips), while simultaneously providing a protective oil barrier to improve moisturizing.

Any numerical values recited herein include all values from the lower value to the upper value in increments of one unit provided that there is a separation of at least 2 units between any lower value and any higher value. As an example, if it is stated that the amount of a component or a value of a process variable such as, for example, temperature, pressure, time and the like is, for example, from 1 to 90, preferably from 20 to 80, more preferably from 30 to 70, it is intended that values such as 15 to 85, 22 to 68, 43 to 51, 30 to 32 etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner. As can be seen, the teaching of amounts expressed as “parts by weight” herein also contemplates the same ranges expressed in terms of percent by weight. Thus, an expression in the Detailed Description of the Invention of a range in terms of at “x parts by weight of the resulting polymeric blend composition” also contemplates a teaching of ranges of same recited amount of “x in percent by weight of the resulting polymeric blend composition.”

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The disclosures of all articles and references, including patent applications and publications, are incorporated by reference for all purposes. The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of, or even consists of, the elements, ingredients, components or steps. By use of the term “may” herein, it is intended that any described attributes that “may” be included are optional.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps. All references herein to elements or metals belonging to a certain Group refer to the Periodic Table of the Elements published and copyrighted by CRC Press, Inc., 1989. Any reference to the Group or Groups shall be to the Group or Groups as reflected in this Periodic Table of the Elements using the IUPAC system for numbering groups.

It will be appreciated that concentrates or dilutions of the amounts recited herein may be employed. In general, the relative proportions of the ingredients recited will remain the same. Thus, by way of example, if the teachings call for 30 parts by weight of a Component A, and 10 parts by weight of a Component B, the skilled artisan will recognize that such teachings also constitute a teaching of the use of Component A and Component B in a relative ratio of 3:1. Teachings of concentrations in the examples may be varied within about 25% (or higher) of the stated values and similar results are expected. Moreover, such compositions of the examples may be employed successfully in the present methods.

It is understood that the above description is intended to be illustrative and not restrictive. Many embodiments as well as many applications besides the examples provided will be apparent to those of skill in the art upon reading the above description. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventors did not It is understood that the above description is intended to be illustrative and not restrictive. Many embodiments as well as many applications besides the examples provided will be apparent to those of skill in the art upon reading the above description.

The explanations and illustrations presented herein are intended to acquaint others skilled in the art with the invention, its principles, and its practical application. Those skilled in the art may adapt and apply the teachings in their numerous forms, as may be best suited to the requirements of a particular use. Accordingly, the specific embodiments of the present teachings as set forth are not intended as being exhaustive or limiting of the teachings. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

1. A composition for forming a solid material at ambient temperature (e.g., 68-77° F.) comprising:
an oil phase including one or more oils in an amount of from 30% to 70% by weight of the composition, and one or more alcohols;
an aqueous phase including one or more polyglycerides;
wherein the ratio of the aqueous phase to the one or more polyglycerides does not exceed 2.5:1.

2. The composition of claim 1, wherein the composition is substantially free of any wax material.

3. (canceled)

4. The composition of claim 1, wherein the oil phase comprises castor oil.

5. The composition of claim 2, wherein the oil phase comprises castor oil in an amount of from about 30% to about 65% by weight of the composition.

6-7. (canceled)

8. The composition of claim 1, wherein the oil phase comprises castor oil in an amount of at least 40% by weight of the composition.

9. The composition of claim 2, wherein the oil phase comprises behenyl alcohol.

10. (canceled)

11. The composition of claim 1, wherein the oil phase comprises behenyl alcohol in an amount of from about 13% to about 25% by weight of the composition.

12. (canceled)

13. The composition of claim 5, wherein the oil phase comprises behenyl alcohol in an amount of at least 15% by weight of the composition.

14. The composition of claim 1, wherein the oil phase comprises hempseed butter in an amount of from about 0.05% to about 20% by weight of the composition.

15-16. (canceled)

17. The composition of claim 1, wherein the oil phase comprises butylated hydroxytoluene (BHT).

18. (canceled)

19. The composition of claim 1, wherein the aqueous phase comprises from about 0.01% to about 30% by weight of the composition.

20-21. (canceled)

22. The composition of claim 1, wherein the aqueous phase comprises less than 20% by weight of the composition.

23. The composition of claim 22, wherein the composition is substantially homogeneous and substantially rigid at temperatures below 50° C.

24. (canceled)

25. The composition of claim 1, wherein the composition has a proximal drop point of at least 55° C.

26. (canceled)

27. The composition of claim 1, wherein the one or more alcohols are selected from C22 fatty alcohol or behenyl alcohol, or combinations thereof.

28-30. (canceled)

31. The composition of claim 1, wherein the aqueous phase includes one or more oleic, linoleic, or linolenic polyglycerides in an amount of from about 0.8% to about 8% by weight of the composition.

32. The composition of claim 2, wherein the aqueous phase includes a non-ionic surfactant/emulsifier selected from sorbitan sesquioleate, Isolan 17 (polyglyceryl-4-diisostearate/polyhydroxystearate/sebacate, caprylic/capric triglyceride, polyglycerides-3-oleate, diisostearyl polyglyceryl-3 dimer dilinoleate, lecithin, or combinations thereof.

33. (canceled)

34. The composition of claim 1, wherein the aqueous phase includes a non-ionic surfactant in an amount of about 0.001% to about 8% by weight of the composition.

35-36. (canceled)

37. The composition of claim 1, wherein the ratio of a non-ionic surfactant to the one or more polyglycerides is from about 1:4 to about 1:10.

38-40. (canceled)

41. The composition of claim 1, comprising: castor oil; behenyl alcohol; hempseed butter and butylated hydroxytoluene.

42-48. (canceled)
